(19) United States
(12) Patent Application Publication
(10) Pub. No.: US 2022/0241250 A1
(43) Pub. Date: Aug. 4, 2022

(54) LIQUID TOPICAL PREPARATION

(71) Applicant: Maruho Co., Ltd., Osaka (JP)

(72) Inventors: Fuminori NARUMI, Osaka (JP); Ayako NAKAMURA, Osaka (JP); Satoko SUZUKI, Osaka (JP)

(21) Appl. No.: 17/262,573

(22) PCT Filed: Jul. 24, 2019
(86) PCT No.: PCT/JP2019/028960

§ 371 Date: Jan. 22, 2021

(30) Foreign Application Priority Data
Jul. 26, 2018 (JP) PCT/JP2018/028049

(51) Int. Cl. A61K 31/436; A61K 9/00; A61K 47/06; A61K 47/08; A61K 47/14

(52) U.S. Cl. CPC A61K 31/436; A61K 9/0014; A61K 47/14; A61K 47/08; A61K 47/06

(57) ABSTRACT

What is aimed at is to provide a topical preparation that contains tacrolimus as an active pharmaceutical ingredient, has high stability and transdermal absorbability of the active pharmaceutical ingredient, is less irritating, and has a good feeling of use. The present invention relates to a liquid topical preparation that contains (i) tacrolimus, a pharmaceutically acceptable salt thereof, or a pharmaceutically acceptable solvate thereof, and (ii) at least one ketone, and that is substantially free of ethanol. It is preferable that the liquid topical preparation further contains at least one fatty acid ester and fluid paraffin, and is substantially free of water.

### TECHNICAL FIELD

The present invention relates to a liquid topical preparation containing tacrolimus, and more particularly to a liquid topical preparation for skin for treating atopic dermatitis and the like.

### BACKGROUND ART

Tacrolimus-containing topical preparations are known to have an excellent therapeutic effect on atopic dermatitis. Currently, Protopic (registered trademark) ointment 0.1% and Protopic (registered trademark) ointment 0.03% for children are used clinically as an oily ointment using an oily base. However, while the oily ointments have merits such as being excellent in skin protection effect, they are difficult to spread when applied to skin, and they are sticky and have a bad feeling of use. So, there is a demand, from patients and medical personnel, for a topical preparation having a better feeling of use.

In contrast, a tacrolimus-containing liquid topical preparation (Tacroz Forte 0.1% Lotion) has been marketed in some countries, but its transdermal absorbability comparable to that of Protopic (registered trademark) ointment has not been confirmed.

In addition, a gel preparation is disclosed in Examples of Patent Document 1, and in Patent Documents 2 and 3, preparation of a lotion and a creamy pharmaceutical composition is attempted. The present applicant has also previously filed an application for a tacrolimus-containing creamy pharmaceutical composition (see Patent Documents 4 and 5). However, it is still desired to develop a composition that has a good feeling of use, is less irritating, and has high stability and transdermal absorbability of an active pharmaceutical ingredient in a preparation.

### PRIOR ART DOCUMENT

### Patent Documents

Patent Document 1: JP-A-2012-149097
Patent Document 2: WO 1994/028894
Patent Document 3: WO 1998/036747
Patent Document 4: WO 2012/011566
Patent Document 5: WO 2013/111817

### SUMMARY OF THE INVENTION

### Problems to be Solved by the Invention

Therefore, an object of the present invention is to provide a tacrolimus-containing pharmaceutical composition (external preparation for skin) that: has a good feeling of use; is less irritating; has high stability of tacrolimus (high residual rate of active pharmaceutical ingredient) in a preparation; and can achieve transdermal absorbability comparable to that of Protopic (registered trademark) ointment.

### Means for Solving the Problems

As a result of repeated experiments to achieve the above object, the present inventors have found that the above object can be achieved by preparing a liquid composition using a ketone as a dissolving agent for tacrolimus and without using ethanol. Thus they have completed the present invention.

That is, the present invention is a liquid topical preparation: that contains (i) tacrolimus, a pharmaceutically acceptable salt thereof, or a pharmaceutically acceptable solvate thereof (hereinafter, they are collectively referred to as tacrolimus), and (ii) at least one ketone; and that is substantially free of ethanol.

By using a ketone as a dissolving agent for tacrolimus, the topical preparation according to the present invention can ensure high transdermal absorbability even without using ethanol, and is excellent in quality and safety. Further, the topical preparation according to the present invention is liquid, so that it has a better feeling of use than ointments.

### Effect of the Invention

According to the present invention, a topical composition, which can stably keep the tacrolimus in a preparation, can exhibit an excellent medicinal effect, is less irritating, and has a good feeling of use (easy to spread and less sticky), can be provided.

### BRIEF DESCRIPTION OF THE DRAWINGS

FIG. 1 is a graph showing the results of in vitro skin permeability tests of tacrolimus for Lotion 1 and Protopic (registered trademark) ointment 0.03%.

FIG. 2 is a graph showing the results of in vitro skin permeability tests of tacrolimus for Lotion 5 and Protopic (registered trademark) ointment 0.1%.

FIG. 3 is a graph showing the results of in vitro skin permeability tests of tacrolimus for Lotion 6 and Protopic (registered trademark) ointment 0.1%.

### MODE FOR CARRYING OUT THE INVENTION

A topical preparation according to the present invention contains tacrolimus as an active pharmaceutical ingredient (API). The content of tacrolimus is preferably 0.01 to 0.3% by weight. If the content of tacrolimus is less than 0.01% by weight, effectiveness will be poor, and if it is more than 0.3% by weight, safety may be impaired. Note that the % by weight of each ingredient described in the present description means the weight percent of each ingredient when the weight of the topical preparation (i.e., the total amount of the preparation) is 100.

As a pharmaceutically acceptable salt of tacrolimus, a non-toxic, pharmaceutically acceptable conventional salt can be used. Examples of such salts include salts with inorganic or organic bases, such as, for example, alkali metal salts (sodium salt, potassium salt, etc.), alkaline earth metal salts (calcium salt, magnesium salt, etc.), ammonium salts, and amine salts (triethylamine salt, N-benzyl-N-methylamine salt, etc.).

Examples of pharmaceutically acceptable solvates of tacrolimus include hydrates and ethanolates.

The topical preparation of the present invention preferably contains a tacrolimus hydrate (particularly, the monohydrate shown below). The tacrolimus monohydrate is well known as the API of Protopic (registered trademark) ointment that is known as a therapeutic agent for atopic dermatitis.

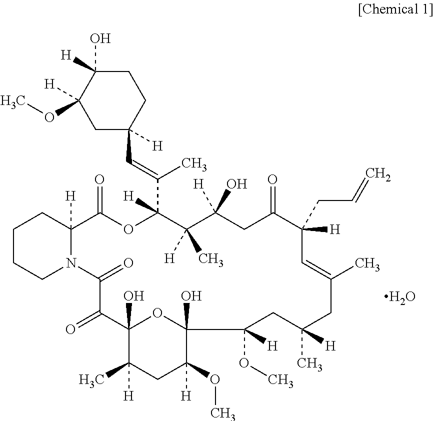

In the topical preparation according to the present invention, a ketone or a plurality of ketones is or are used as a dissolving agent for tacrolimus. When used as a dissolving agent for tacrolimus, ketone exhibits a high partition coefficient K between preparation and skin, and thus a topical preparation excellent in transdermal absorbability of tacrolimus can be provided. Further, the tacrolimus in the topical preparation can be stably kept by using a ketone as a dissolving agent.

Preferred examples of the ketone include ketones represented by the formula: R—(C═O)—R′ where R and R′ are alkyl groups having 1 to 4 carbon atoms, respectively. The alkyl group may be linear or branched. In particular, a ketone in which either R or R′ is a methyl group is more preferable. A more preferred ketone is a ketone selected from the group consisting of methyl ethyl ketone, acetone, and methyl isobutyl ketone. A particularly preferred ketone is a ketone selected from the group consisting of methyl ethyl ketone and methyl isobutyl ketone. The most preferred ketone is a methyl ethyl ketone. The ketone may be a ketone other than acetone. It is preferable that the ketone is not a ketone to be used as a viscosity increasing agent such as polyethylene pyrrole ketone.

The total content of the ketones in the topical preparation is preferably 3 to 15% by weight. If below 3% by weight, the stability and absorbability of tacrolimus are impaired. A more preferred content is 4 to 12% by weight, a particularly preferred content is 4.5 to 11% by weight, and an even more preferred content is 5 to 10% by weight.

The topical preparation of the present invention is substantially free of ethanol. Being substantially free of ethanol means that ethanol is not intentionally added in the manufacturing process. Therefore, the ethanol content of the topical preparation of the present invention is usually 0% by weight, and even though a very small amount of ethanol is present therein, the ethanol content is less than 1% by weight (more preferably less than 0.5% by weight). Tacrolimus is easily soluble in ethanol, and ethanol exhibits a high partition coefficient K of tacrolimus, but ethanol is known to be irritating to skin. Considering that tacrolimus-containing topical preparations are used for treating atopic dermatitis (i.e., applied to the skin with impaired barrier function), it is preferable that they are free of ethanol that is irritating to skin. Since the topical preparation of the present invention is substantially free of ethanol, it is less irritating to skin and is suitable for treating atopic dermatitis.

It is further preferable that the present invention is substantially free of lower monohydric alcohols (monohydric alcohols having 1 to 3 carbon atoms, e.g., isopropanol) other than ethanol.

It is preferable that the topical preparation of the present invention is non-aqueous, that is, substantially free of water. Being substantially free of water means that water is not intentionally added in the manufacturing process. Therefore, when the topical preparation according to the present invention is non-aqueous, the water content thereof is usually less than 1% by weight (more preferably less than 0.5% by weight, and particularly preferably 0% by weight).

In addition, the topical preparation of the present invention may be substantially free of hydrophilic polymers (carboxyvinyl polymer, etc.). In addition, the topical preparation of the present invention may be substantially free of a surfactant. In addition, the topical preparation of the present invention may be substantially free of polyhydric alcohols (e.g., glycerin, propylene glycol, 1,3-butylene glycol). In addition, the topical preparation of the present invention may be an oily preparation, for example, an oily lotion, which is substantially free of aqueous ingredients (water and ingredients to be miscible with water, such as polyhydric alcohols and lower monohydric alcohols). In addition, the topical preparation of the present invention may be substantially free of acetone. Being substantially free of an ingredient means that the content thereof is less than 1% by weight (more preferably less than 0.5% by weight, and particularly preferably 0% by weight).

The topical preparation of the present invention preferably further contains at least one fatty acid ester. By using a ketone and a fatty acid ester in combination, transdermal absorbability can be improved while maintaining the stability of tacrolimus.

Examples of the fatty acid ester to be used in the present invention include fatty acid monoesters, fatty acid diesters, and glycerin fatty acid esters.

Examples of such fatty acid esters include esters of saturated or unsaturated fatty acids having 6 to 22 carbon atoms (preferably 8 to 20, and more preferably 8 to 18) (e.g., caproic acid, caprylic acid, 2-ethylhexanoic acid, isooctanoic acid, capric acid, lauric acid, myristic acid, palmitic acid, stearic acid, isostearic acid, oleic acid, behenic acid, isostearic acid) and alcohols having 1 to 22 carbon atoms (preferably 3 to 20) (e.g., methanol, ethanol, propanol, isopropanol, glycerol, 1-decanol, 1-dodecanol, 1-tetradecanol, cetanol, 1-hexadecanol, stearyl alcohol, isostearyl alcohol, cetostearyl alcohol, octyldodecyl alcohol, behenyl alcohol).

Preferred examples of the fatty acid esters include a fatty acid ester or a plurality of fatty acid esters selected from the group consisting of octyldodecyl myristate, hexadecyl isostearate, isopropyl palmitate, cetyl 2-ethylhexanoate, isopropyl myristate, decyl oleate, and medium-chain triglycerides (e.g., glycerin triisooctanoate and tri(caprylic/capric acid) glycerin, etc.).

The fatty acid esters may be used alone or in combination of two or more types. Examples of the preferred topical preparations of the present invention include topical preparations containing 2 or 3 types of fatty acid esters selected from the group consisting of hexadecyl isostearate, octyldodecyl myristate, diethyl sebacate, and medium-chain triglycerides. A particularly preferred example includes a topical preparation containing hexadecyl isostearate and octyldodecyl myristate, or a topical preparation containing hexadecyl isostearate, octyldodecyl myristate, and medium-chain triglyceride.

The total content of at least one fatty acid ester in the topical preparation of the present invention is preferably 15 to 50% by weight, more preferably 20 to 45% by weight, particularly preferably 23 to 42% by weight, and even more preferably 25 to 40% by weight.

The topical preparation of the present invention preferably further contains at least one fluid paraffin such as light liquid paraffin (light mineral oil) or liquid paraffin (mineral oil). The content of the fluid paraffin in the topical preparation of the present invention is preferably 45 to 80% by weight, more preferably 50 to 75% by weight, particularly preferably 52 to 72% by weight, and even more preferably 53 to 70% by weight.

In the topical preparation of the present invention, the ratio of the at least one fatty acid ester to the at least one ketone, that is, the total weight of the fatty acid ester:the total weight of the ketone, is preferably within the range of 2.5 to 10:1, more preferably within the range of 2.8 to 9:1, and particularly preferably within the range of 3 to 8:1.

In particular, when the ketone is only methyl ethyl ketone and the fatty acid esters are two types of hexadecyl isostearate and octyldodecyl myristate, the total weight of hexadecyl isostearate and octyldodecyl myristate is preferably within the range of 3 to 8, and more preferably within the range of 4 to 6, based on 1 of the weight of methyl ethyl ketone.

In the topical preparation of the present invention, the ratio of the fluid paraffin to the at least one ketone, that is, the total weight of the fluid paraffin:the total weight of the ketone, is preferably within the range of 5 to 15:1.

The topical preparation according to the present invention is in a liquid state (e.g., a lotion), and its viscosity is preferably generally 2000 mPa·s or less, more preferably 1000 mPa·s or less, and particularly preferably 200 mPa·s or less. In the present invention, the viscosity means a viscosity obtained when measurement is performed using a cone-plate type viscometer (MCR302, jig CP50-1) at a measurement temperature of 25° C., at rotation speed of 5 rpm, and for 45 seconds.

The topical preparation according to the present invention may contain a preservative and the like in addition to the above ingredients.

Examples of the preservative include methyl paraoxybenzoate, ethyl paraoxybenzoate, propyl paraoxybenzoate, sodium benzoate, phenoxyethanol, and the like. The preservatives may be used alone or in combination of two or more. The content of the preservative in the topical preparation is preferably within the range of 0.01 to 2% by weight, and more preferably within the range of 0.1 to 1.0% by weight. If the content is more than 2% by weight, the safety of a preparation may be impaired.

One preferred example of the topical preparation of the present invention includes a liquid preparation: that contains (i) tacrolimus hydrate, (ii) 3 to 15% by weight of at least one ketone, (iii) 15 to 50% by weight of at least one fatty acid ester, and (iv) 45 to 80% by weight of at least one fluid paraffin; and that is substantially free of water and ethanol.

In addition, one preferred example of the topical preparation of the present invention includes a liquid preparation: that contains (i) tacrolimus hydrate, (ii) methyl ethyl ketone, (iii) 2 or 3 types of fatty acid esters selected from hexadecyl isostearate, octyldodecyl myristate, diethyl sebacate, and medium-chain triglyceride, and (iv) fluid paraffin; and that is substantially free of water and ethanol.

In addition, a more preferred example of the topical preparation of the present invention includes a liquid preparation: that contains (i) tacrolimus hydrate, (ii) 3 to 15% by weight (more preferably 4 to 12% by weight) of methyl ethyl ketone, (iii) a total of 15 to 50% by weight (more preferably 20 to 45% by weight) of 2 or 3 types of fatty acid esters selected from hexadecyl isostearate, octyldodecyl myristate, and medium-chain triglyceride, and (iv) 45 to 80% by weight (more preferably 50 to 75% by weight) of fluid paraffin; and that is substantially free of water and ethanol.

In addition, a particularly preferred example of the topical preparation of the present invention includes a liquid preparation: that contains (i) tacrolimus hydrate, (ii) 3 to 15% by weight (more preferably 4 to 12% by weight) of methyl ethyl ketone, (iii) 5 to 10% by weight of hexadecyl isostearate, 15 to 20% by weight of octyldodecyl myristate, and 0 to 20% by weight of medium-chain triglyceride, and (iv) 45 to 75% by weight (more preferably 50 to 73% by weight) of fluid paraffin; and that is substantially free of water and ethanol.

In the topical preparation of the present invention, the total content of the ingredients other than the above (i) to (iv) is preferably 10% by weight or less, more preferably 5% by weight or less, and particularly preferably 3% by weight or less.

The topical preparation of the present invention is useful for treating allergic diseases of the skin with atopic dermatitis or the like.

The amount and frequency of application, to the skin, of the topical preparation according to the present invention may be appropriately adjusted according to the symptom of the skin, the concentration of the tacrolimus in the topical preparation, the age of a patient, and the like. It is usually appropriate to apply once or twice a day. It is preferable that the topical preparation of the present invention is not used for the eye (particularly, the anterior part of the eye and the surface of the eye).

In the preceding paragraphs, the preferred compound names of the essential and optional ingredients to be used in the topical preparation of the present invention have been described. The topical preparation of the present invention includes topical preparations obtained by arbitrarily combining them. Also included are topical preparations that are obtained by arbitrarily combining the concentration ranges described for the respective ingredients. In addition, the ranges of values such as concentration and viscosity values described in the preceding paragraphs can also be arbitrarily combined, and when a plurality of ranges are described, the upper limit values or the lower limit values of the respective ranges can also be arbitrarily combined.

Hereinafter, the present invention will be described in more detail with reference to examples, but the present invention is not limited to the examples. The tacrolimus used in the examples are all tacrolimus monohydrate (obtained from Astellas Pharma Inc.), and in the following examples, they are simply referred to as tacrolimus or tacrolimus hydrate.

### EXAMPLES

### [Example 1] In Vitro Hairless Mouse Skin Permeability Test

A skin permeability experiment was performed using a preparation (liquid) obtained by dissolving tacrolimus hydrate in each of the dissolving agents shown in Table 1.

First, the thickness of hairless mouse skin (thickness of a preparation application site) naturally thawed at room temperature was measured. The hairless mouse skin was set in a Franz vertical permeation cell, and 1 mL of each preparation was applied. An in vitro transdermal absorption automatic sampling system was used to collect a receptor liquid in the Franz vertical permeation cell at specified sampling times. The concentration of tacrolimus in the collected receptor liquid was measured using a liquid chromatography tandem mass spectrometer (LC/MS/MS). Based on the results, the partition coefficient K between preparation and skin was calculated as an index of the concentration of tacrolimus in the skin. A calculation formula for the partition coefficient K is as follows (where, the lag time is a time required for a skin permeation rate to reach a steady state).

$$K = \frac{6 \times Permeation rate ( ng / {cm}^{2} / hr ) \times Lag time ( hr )}{\begin{matrix}Skin thickness ( cm ) \times Concentration \\ of tacrolimus in prepration ( ng / {cm}^{3} )\end{matrix} } \qquad [ Formula 1 ]$$

The results are shown in Table 1. The higher the partition coefficient K, the higher the concentration of tacrolimus in the skin (i.e., the higher the partition coefficient K of a solvent, the easier it is for tacrolimus to migrate from the solvent to the skin).

### [Example 2] Stability Test

The prepared preparation was stored under predetermined conditions for a certain period of time, and the content of tacrolimus hydrate was measured according to the “17th Revised Japanese Pharmacopoeia General Test Method Liquid Chromatography <2.01>”, and the residual rate of tacrolimus hydrate in the liquid composition was calculated by the following formula, thereby evaluating the stability of the API in the composition.

$$Residual rate of tacrolimus hydrate ( \% ) = \frac{\begin{matrix}Content of tacrolimus hydrate in \\ stored preparation ( \% )\end{matrix}}{\begin{matrix}Content of tacrolimus hydrate in \\ initial preparation ( \% )\end{matrix}} \times 100 \qquad [ Formula 2 ]$$

The results are shown in Table 1.

TABLE 1

| | | | Residual |
|---|---|---|---|
| | | Partition | rate of |
| | | coefficient | API |
| | | K | (60° C./ |
| | Solvent | (×10⁻³) | 1 W) |
| No. 1 | Methyl ethyl ketone | 153 | 98.7% |
| No. 2 | Sorbitan sesquioleate | 0.69 | — |
| No. 3 | Polyoxyethylene sorbitan | 6.08 | — |
| | trioleate | | |
| No. 4 | Polyoxyethylene oleyl ether | 0.51 | — |
| No. 5 | Polysorbate 80 | 6.59 | — |
| No. 6 | Polypropylene glycol 2000 | 0.07 | 95.5% |
| No. 7 | Hexyl laurate | 3.76 | — |
| No. 8 | Diethylene glycol monoethyl | 0.36 | 73.3% |
| | ether | | |
| No. 9 | Dimethyl isosorbide | 0.09 | 92.9% |
| No. 10 | Ethyl oleate | 0.89 | 98.9% |
| No. 11 | Hexylene glycol | 0.06 | 94.3% |
| No. 12 | Isopropyl isostearate | 0.84 | 98.0% |
| No. 13 | Poloxamer 181 | 0.04 | 77.7% |
| No. 14 | PPG-11 stearyl ether | 0.41 | 81.5% |
| No. 15 | Triacetin | 0.05 | 93.3% |
| Reference | Ethanol | 2496 × 10³ | — |
| example | | | |

As is clear from Table 1, it was found that methyl ethyl ketone (No. 1) has an extremely higher ability to partition tacrolimus to skin than the other solvents (No. 2 to No. 15). Methyl ethyl ketone (No. 1) also exhibited a high residual rate of the API (tacrolimus). On the other hand, ethanol exhibited a much higher partition coefficient than methyl ethyl ketone. However, considering that tacrolimus is used for treating atopic dermatitis, the skin irritation of ethanol is expected to adversely affect the skin of a patient with impaired barrier function. Therefore, a preparation, which has high transdermal absorbability without using ethanol, was aimed to be developed.

### [Example 3] In Vitro Hairless Mouse Skin Permeability Test and Stability Test

Next, a solvent, which can improve transdermal absorbability while maintaining the stability of tacrolimus by being used in combination with methyl ethyl ketone (MEK), was evaluated. As a result, it was found that by using methyl ethyl ketone and fatty acid esters in combination, the partition of tacrolimus to the skin can further be enhanced and the stability of tacrolimus in the preparation can be maintained, as shown in Table 2. The results are shown in Table 2. The % of the solvents in Table 2 means % by weight. The partition coefficient K and the residual rate of the API were determined using the same methods as in Examples 1 and 2, respectively.

TABLE 2

| | | | Residual |
|---|---|---|---|
| | | Partition | rate of |
| | | coefficient | API |
| | | K | (60° C./ |
| | Solvent | (×10⁻³) | 1 W) |
| No. 16 | MEK (82%) + Octyldodecyl myris- | 1176 | 99.4% |
| | tate (18%) | | |
| No. 17 | MEK (90%) + Isopropyl palmitate | 452 | 98.5% |
| | (10%) | | |
| No. 18 | MEK (85%) + Hexadecyl isostearate | 746 | 99.3% |
| | (15%) | | |
| No. 19 | MEK (97%) + Cetyl 2-ethylhexanoate | 5824 | 98.6% |
| | (3%) | | |
| No. 20 | MEK (90%) + Tri(caprylic/capric acid) | 159 | 98.7% |
| | glycerin (10%) | | |
| No. 21 | MEK (95%) + Isopropyl myristate (5%) | 289 | 99.1% |
| No. 22 | MEK (95%) + Decyl oleate (5%) | 1836 | 99.3% |
| No. 23 | MEK (95%) + Glycerin triisooctanoate | 203 | 99.1% |
| | (5%) | | |

### [Example 4] In Vitro Hairless Mouse Skin Permeability Test

In order to compare the partition coefficients K of the preparation containing the dissolving agent (triacetin) for tacrolimus disclosed in Patent Document 1 (JP-A-2012-149097) and that of a preparation containing methyl ethyl ketone, the following compositions were produced by referring to Preparation Example 3 of Patent Document 1. And, the partition coefficient K was determined by the same method as in Example 1.

The results are shown in Table 3. The numerical value of each ingredient in Table 3 represents % by weight.

TABLE 3

| Ingredient name | Composition No. 1 | Composition No. 2 |
|---|---|---|
| Tacrolimus hydrate | 0.031 | 0.031 |
| Methyl ethyl ketone | 5 | — |
| Triacetin | — | 5 |
| Glyceryl triethylhexanoate | 5 | 5 |
| Dextrin palmitate | 5 | 5 |
| Liquid paraffin (Mineral oil) | balance | balance |
| Total amount | 100 | 100 |
| Partition coefficient K (×10⁻³) | 128 | 18 |

As shown in Table 3, Composition No. 1 containing methyl ethyl ketone exhibits a much higher partition coefficient than Composition No. 2 containing triacetin, so that a methyl ethyl ketone-containing preparation can be expected to be more excellent in transdermal absorbability of tacrolimus than a triacetin-containing preparation. The partition coefficient K for the tacrolimus-containing liquid topical preparation “Tacroz Forte 0.1% Lotion” currently on the market in India was also determined by the same method and found to be 16.28×10⁻³. Therefore, Composition No. 1 can be expected to be more excellent in transdermal absorbability of tacrolimus even than Tacroz Forte 0.1% Lotion.

### [Example 5] Prescription Example

In consideration of absorbability, quality, and safety, the topical preparations shown in Tables 4 and 5 were prepared by using methyl ethyl ketone and other ingredients. Each topical preparation was prepared by dissolving tacrolimus hydrate in methyl ethyl ketone and then adding the other ingredients thereto and mixing them uniformly.

### [Example 6] In Vitro Hairless Mouse Skin Permeability Test and Stability Test

For each of the topical preparations shown in Tables 4 and 5, transdermal absorbability (after 16 hours and after 24 hours) and stability (60° C. for 2 weeks) were confirmed.

Transdermal absorbability (after 16 hours or after 17 hours and after 24 hours) tests were performed using the same method as in Example 1. More specifically, for each Lotion in Tables 4 and 5 and each of Protopic (registered trademark) ointments 0.03% and 0.1%, the cumulative permeation amounts of tacrolimus were calculated, and then the transdermal absorbability for the preparation containing 0.031% of tacrolimus monohydrate was represented by the ratio of the cumulative permeation amount for it to the cumulative permeation amount for Protopic (registered trademark) ointment 0.03%, and the transdermal absorbability for the preparation containing 0.102% of tacrolimus monohydrate was represented by the ratio of the cumulative permeation amount for it to the cumulative permeation amount for Protopic (registered trademark) ointment 0.1%.

A stability test was performed using the same method as in Example 2.

The results are shown in Tables 4 and 5. The numerical value of each ingredient in Tables 4 and 5 represents % by weight. When the viscosity of Lotion 1 was measured (cone-plate type viscometer (MCR302, jig CP50-1), at a measurement temperature of 25° C., at rotation speed of 5 rpm, and for 45 seconds), it was about 50 mPa·s.

TABLE 4

| | Lo- | Lo- | Lo- | Lo- |
|---|---|---|---|---|
| | tion 1 | tion 2 | tion 3 | tion 4 |
| Tacrolimus hydrate | 0.031 | 0.031 | 0.031 | 0.031 |
| Methyl ethyl ketone | 5 | 5 | 5 | 5 |
| Hexadecyl isostearate | 7 | 11 | 15 | 15 |
| Octyldodecyl myristate | 18 | 14 | 10 | 14 |
| Diethyl sebacate | — | — | — | — |
| Medium-chain triglyceride | — | — | — | — |
| Liquid paraffin (Mineral oil) | balance | balance | balance | balance |
| Total amount | 100 | 100 | 100 | 100 |
| Transdermal absorbability (16 h) | 109% | 122% | 146% | 163% |
| Transdermal absorbability (24 h) | 86% | 94% | 110% | 120% |
| Residual rate of API (60° C./ | 97.6% | 97.0% | 97.5% | 97.1% |
| 2 W) | | | | |

TABLE 5

| | Lotion 5 | Lotion 6 | Lotion 7 | Lotion 8 | Lotion 9 | Lotion 10 | Lotion 11 |
|---|---|---|---|---|---|---|---|
| Tacrolimus hydrate | 0.102 | 0.102 | 0.102 | 0.102 | 0.102 | 0.102 | 0.102 |
| Methyl ethyl ketone | 5 | 10 | 5 | 10 | 10 | 7 | 10 |
| Hexadecyl isostearate | 7 | 7 | — | — | 7 | 7 | 7 |
| Octyldodecyl myristate | 18 | 18 | 10 | 10 | 18 | 18 | 18 |
| Diethyl sebacate | — | — | 15 | — | 5 | — | — |
| Medium-chain triglyceride | 15 | 7 | — | 15 | — | 15 | 15 |
| Liquid paraffin (Mineral oil) | balance | balance | balance | balance | balance | balance | balance |
| Total amount | 100 | 100 | 100 | 100 | 100 | 100 | 100 |
| Transdermal absorbability (17 h) | 122% | 114% | 147% | 114% | 103% | 143% | 132% |
| Transdermal absorbability (24 h) | 91% | 87% | 111% | 82% | 74% | 104% | 99% |
| Residual rate of API (60° C./2 W) | 98.5% | 98.4% | 100.3% | 99.8% | 99.5% | 98.2% | 98.4% |

As shown in Tables 4 and 5, all of Lotions 1 to 11 exhibited high transdermal absorbability and residual rate of the API. In particular, Lotions 1, 2, 5, 6, and 8 exhibited transdermal absorbability approximate to Protopic (registered trademark) ointment (within 100±25%).

### [Example 7] Permeation Profile

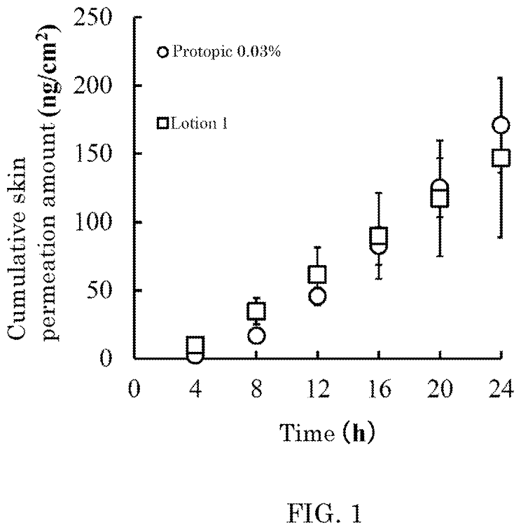

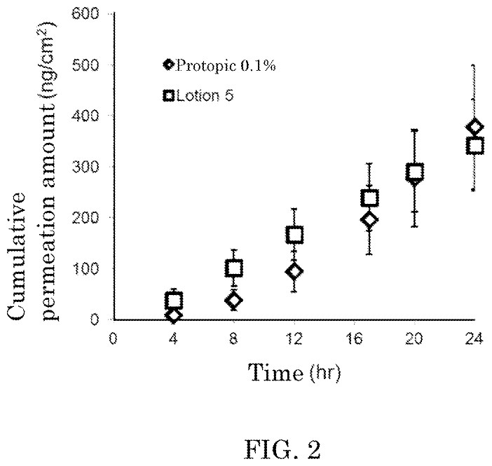

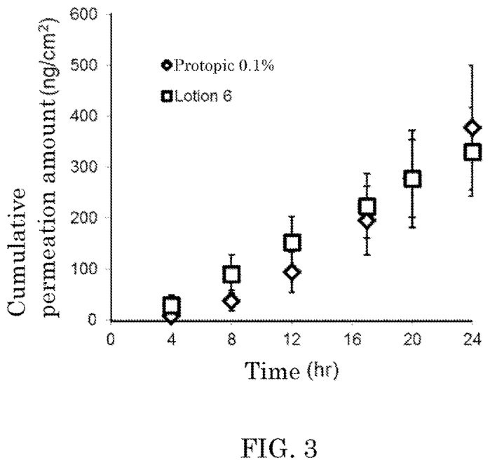

The skin permeation profile of the topical preparation of the present invention was compared with the permeation profile of Protopic (registered trademark) ointment that has already been used clinically. More specifically, for Lotions 1, 5, and 6 in Table 4 and Protopic (registered trademark) ointments 0.03% and 0.1%, in vitro hairless mouse skin permeability tests were performed in the same way as described above, and the transdermal absorbability of tacrolimus (cumulative permeation amount of tacrolimus) was measured every 4 hours and compared. The results are shown in FIGS. 1 to 3.

As shown in FIG. 1, Lotion 1 exhibited a permeation profile approximate to Protopic (registered trademark) ointment 0.03%. Lotions 5 and 6 exhibited a permeation profile approximate to Protopic (registered trademark) ointment 0.1%. Therefore, equivalence is expected to be established between these Lotions and Protopic (registered trademark) ointments.

### [Example 8] Stability Test

Each of Lotions 1, 5, and 6 shown in Table 4 and Cream 1 shown in Table 6 below was put in a container (glass vial, aluminum tube, or plastic bottle), and stored under harsh conditions (40° C./75% RH) for a predetermined period.

At a predetermined time, a measurement sample was taken out of each preparation, and by using a liquid chromatography, the peak areas Ai of a peak of tacrolimus, a peak at a relative retention time of about 0.7 for tacrolimus (related substance A), a peak at a relative retention time of about 0.8 for tacrolimus (related substance B), and a peak at a relative retention time of about 0.85 for tacrolimus (related substance C) were measured by an automatic analysis method. Thereby, the ratios of the related substances (substances caused by the decomposition of tacrolimus) were determined. Operating conditions of the liquid chromatography were as follows.

Operating Conditions

Detector: Ultraviolet absorptiometer (measurement wavelength: 225 nm)

Column: Two stainless steel tubes with an inner diameter of 4.6 mm and a length of 25 cm, which were filled with 5-μm dihydropropyl silylated silica gel for liquid chromatography, were connected.

Mobile phase: Hexane/n-butyl chloride/acetonitrile mixture (7:2:1)

Column temperature: Constant temperature around 28° C.

Flow rate: Adjusted such that the retention time of tacrolimus was about 15 minutes. (Approximately 1.5 mL/min)

Table 6 shows the composition of a comparative preparation (cream preparation), and Table 7 shows the results of the stability test. The value of each ingredient in Table 6 represents % by weight.

TABLE 6

| | Cream 1 |
|---|---|
| Tacrolimus hydrate | 0.102 |
| Diisopropyl adipate | 10 |
| PEG-60 hydrogenated castor oil | 2 |
| Dibutylhydroxytoluene | 0.1 |
| Propyl paraoxybenzoate | 0.05 |
| 1,3-Butylene glycol | 10 |
| Methyl paraoxybenzoate | 0.2 |
| Sodium edetate hydrate | 0.02 |
| Carboxyvinyl polymer | 1 |
| Diisopropanolamine | 0.5 |
| Purified water | balance |
| Total amount | 100 |

TABLE 7

| | | Period | | | |
|---|---|---|---|---|---|
| | | | 1 | 3 | 6 |
| | Name | Initial | month | months | months |
| Lotion 1 | Related substance A | <0.05% | <0.05% | 0.08% | <0.05% |
| | Related substance B | <0.10% | <0.10% | <0.10% | <0.10% |
| | Related substance C | <0.05% | <0.05% | <0.05% | <0.05% |
| | Others | <0.05% | <0.05% | <0.05% | <0.05% |
| | Total amount | <0.05% | <0.05% | 0.07% | 0.14% |
| Lotion 5 | Related substance A | <0.05% | <0.05% | <0.05% | <0.05% |
| | Related substance B | <0.10% | <0.10% | <0.10% | 0.24% |
| | Related substance C | <0.05% | <0.05% | <0.05% | 0.09% |
| | Others | <0.05% | <0.05% | <0.05% | <0.05% |
| | Total amount | <0.05% | <0.05% | 0.08% | 0.33% |
| Lotion 6 | Related substance A | <0.05% | <0.05% | <0.05% | <0.05% |
| | Related substance B | <0.10% | <0.10% | <0.10% | 0.19% |
| | Related substance C | <0.05% | <0.05% | <0.05% | 0.09% |
| | Others | <0.05% | <0.05% | <0.05% | <0.05% |
| | Total amount | <0.05% | <0.05% | <0.05% | 0.28% |
| Cream 1 | Related substance A | <0.05% | 0.09% | 0.42% | 0.82% |
| | Related substance B | <0.10% | <0.10% | 0.35% | 0.57% |
| | Related substance C | <0.05% | 0.20% | 0.71% | 1.52% |
| | Others | <0.05% | <0.05% | <0.05% | <0.05% |
| | Total amount | <0.05% | 0.29% | 1.51% | 2.99% |

As shown in Table 7, the lotion preparation according to the present invention has demonstrated that the related substances caused by the decomposition of tacrolimus are significantly smaller in amount than the cream preparation and the stability of tacrolimus in the preparation is high even when stored at room temperature. The fact that the stability of tacrolimus in Cream 1 is low is most likely to be caused by the water contained in the cream preparation, and hence it is preferable that the liquid topical preparation of the present invention is free of water.

### [Example 8] Skin Irritation Test

For Lotions 1, 2, 5, 6, and their respective placebos (Placebos 1, 2, 5, 6), cumulative skin irritation was evaluated using female rabbits (Kbl: NZW) (for Lotions 1, 2 and their placebos, n=3, for Lotions 5, 6 and their placebos, n=4). Four administration sites (damaged skin) each having a size of 2.5×2.5 cm, per one rabbit, were provided on the depilated back skin of the rabbit, so that the above lotions were applied openly at 0.05 mL/site for about 23 hours a day for 14 consecutive days. A skin reaction was determined according to the criteria of Draize, J. H. (Appraisal of the safety of chemicals in foods, drugs and cosmetics, The Association of Food and Drug Officials of the United States, Topeka, Kans., 46-59, 1965). More specifically, erythema, crust formation, and edema formation were respectively scored with a score of 0 to 4, and the degree of cumulative skin irritation was evaluated based on the following determination criteria.

The results are shown in Table 8.

TABLE 8

| Day of | Total average score ^{a)} | | | | | | | |
|---|---|---|---|---|---|---|---|---|
| observation | Lotion 1 | Lotion 2 | Lotion 5 | Lotion 6 | Placebo 1 | Placebo 2 | Placebo 5 | Placebo 6 |
| 1 | 0.7 | 0.7 | 0.3 | 0.3 | 0.7 | 0.7 | 0.3 | 0.3 |
| 2 | 0.7 | 0.7 | 0.3 | 0.0 | 0.7 | 0.7 | 0.3 | 0.3 |
| 3 | 0.0 | 0.0 | 0.0 | 0.0 | 0.0 | 0.0 | 0.0 | 0.3 |
| 4 | 0.0 | 0.0 | 0.0 | 0.0 | 0.0 | 0.0 | 0.0 | 0.0 |
| 5 | 0.0 | 0.0 | 0.0 | 0.0 | 0.0 | 0.0 | 0.0 | 0.0 |
| 6 | 0.0 | 0.0 | 0.0 | 0.0 | 0.0 | 0.0 | 0.0 | 0.0 |
| 7 | 0.0 | 0.0 | 0.0 | 0.0 | 0.0 | 0.0 | 0.0 | 0.0 |
| 8 | 0.0 | 0.0 | 0.0 | 0.0 | 0.0 | 0.0 | 0.0 | 0.0 |
| 9 | 0.0 | 0.0 | 0.0 | 0.0 | 0.0 | 0.0 | 0.0 | 0.0 |
| 10 | 0.0 | 0.0 | 0.0 | 0.0 | 0.0 | 0.0 | 0.0 | 0.0 |
| 11 | 0.0 | 0.0 | 0.0 | 0.0 | 0.0 | 0.0 | 0.0 | 0.0 |
| 12 | 0.0 | 0.0 | 0.0 | 0.0 | 0.0 | 0.0 | 0.0 | 0.0 |
| 13 | 0.0 | 0.0 | 0.0 | 0.0 | 0.0 | 0.0 | 0.0 | 0.0 |
| 14 | 0.0 | 0.0 | 0.0 | 0.0 | 0.0 | 0.0 | 0.0 | 0.0 |
| Average score ^{b)} | 0.1 | 0.1 | 0.0 | 0.0 | 0.1 | 0.1 | 0.0 | 0.1 |
| Determination ^{c)} | Weak | Weak | Weak | Weak | Weak | Weak | Weak | Weak |
| | irritant | irritant | irritant | irritant | irritant | irritant | irritant | irritant |

^{a)} Total average score = Σ (score of erythema and crust formation + score of edema formation)/number of animals
^{b)} Average score = Σ (total average score)/number of observation days
^{c)} Evaluation of irritation

As can be seen from Table 8, each of Lotions 1, 2, 5, and 6 and Placebos 1, 2, 5, and 6 had an average score of 0 or 0.1, and was classified into the least irritating category (weak irritant).

From the results of Examples 1 to 8, it has been found that the topical preparation of the present invention exhibits a permeation profile of tacrolimus approximate to that of Protopic (registered trademark) ointment which has already been used clinically, and that it has high stability of the tacrolimus in the preparation and is less irritating. Further, the topical preparation of the present invention is a liquid, so that it can be more easily spread on the skin than an ointment, and is less sticky and shiny. Therefore, according to the present invention, a tacrolimus-containing preparation, which has a good feeling of use, has high absorbability of the API, and is excellent in quality and stability, can be provided.

1. A liquid topical preparation that contains (i) tacrolimus, a pharmaceutically acceptable salt of the tacrolimus, or a pharmaceutically acceptable solvate of the tacrolimus, and (ii) at least one ketone, and that is substantially free of ethanol.

2. The liquid topical preparation according to claim 1, wherein the at least one ketone is selected from the group consisting of methyl ethyl ketone and methyl isobutyl ketone.

3. The liquid topical preparation according to claim 1, wherein the at least one ketone is selected from ketones other than acetone.

4. The liquid topical preparation according to claim 1, wherein the at least one ketone is methyl ethyl ketone.

5. The liquid topical preparation according to claim 1, being substantially free of water.

6. The liquid topical preparation according to claim 1, further containing at least one fatty acid ester.

7. The liquid topical preparation according to claim 6, wherein the at least one fatty acid ester is a fatty acid ester or a plurality of fatty acid esters selected from the group consisting of octyldodecyl myristate, hexadecyl isostearate, isopropyl palmitate, cetyl 2-ethylhexanoate, isopropyl myristate, decyl oleate, and medium-chain triglyceride.

8. The liquid topical preparation according to claim 1, further containing at least one fluid paraffin.

9. The liquid topical preparation according to claim 1 that contains (i) tacrolimus hydrate, (ii) 3 to 15% by weight of at least one ketone, (iii) 15 to 50% by weight of at least one fatty acid ester, and (iv) 45 to 80% by weight of at least one fluid paraffin, and that is substantially free of water and ethanol.

10. The liquid topical preparation according to claim 1 that contains (i) tacrolimus hydrate, (ii) methyl ethyl ketone, (iii) two or three types of fatty acid esters selected from hexadecyl isostearate, octyldodecyl myristate, diethyl sebacate, and medium-chain triglyceride, and (iv) fluid paraffin, and that is substantially free of water and ethanol.

11. The liquid topical preparation according to claim 1 that contains (i) tacrolimus hydrate, (ii) 3 to 15% by weight of methyl ethyl ketone, (iii) a total of 15 to 50% by weight of two or three types of fatty acid esters selected from hexadecyl isostearate, octyldodecyl myristate, and medium-chain triglyceride, and (iv) 45 to 80% by weight of fluid paraffin, and that is substantially free of water and ethanol.

12. The liquid topical preparation according to claim 1 that contains (i) tacrolimus hydrate, (ii) 3 to 15% by weight of methyl ethyl ketone, (iii) 5 to 10% by weight of hexadecyl isostearate, 15 to 20% by weight of octyldodecyl myristate, and 0 to 20% by weight of medium-chain triglyceride, and (iv) 45 to 75% by weight of fluid paraffin, and that is substantially free of water and ethanol.

13. The liquid topical preparation according to claim 1 that is a preparation for skin application to be used for treating an allergic disease of skin.

14. The liquid topical preparation according to claim 13 that is substantially free of acetone.
